SEQUENCE LISTING (20 sequences)

SEQ ID NO: 1 - protein, 1267 aa, Severe acute respiratory syndrome-related coronavirus
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1276 aa, Severe acute respiratory syndrome-related coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVEPERDLPQGFSALEPLVDLPIGINITRFQTLLALHRSYLTPGDSSSGWTAGAAAYYVGYLQPMTYILKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECINLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 1113 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVEPERDLPQGFSALEPLVDLPIGINITRFQTLLALHRSYLTPGDSSSGWTAGAAAYYVGYLQPMTYILKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECINLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFY

SEQ ID NO: 4 - protein, 98 aa, synthetic construct
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNI

SEQ ID NO: 5 - protein, 97 aa, synthetic construct
IRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGY

SEQ ID NO: 6 - protein, 106 aa, synthetic construct
FKIYSKHTPIIVEPERDLPQGFSALEPLVDLPIGINITRFQTLLALHRSYLTPGDSSSGWTAGAAAYYVGYLQPMTYILKYNENGTITDAVDCALDPLSETKCTLK

SEQ ID NO: 7 - protein, 96 aa, synthetic construct
SFTVEKGIYQTSNFRVQPTESIVRFPNITNLCPFDEVFNATRFASVYAWNRKRISNCVADYSVLYNLAPFFTFKCYGVSPTKLNDLCFTNVYADSF

SEQ ID NO: 8 - protein, 110 aa, synthetic construct
VIRGDEVRQIAPGQTGNIADYNYKLPDDFTGCVIAWNSNKLDSKVSGNYNYLYRLFRKSNLKPFERDISTEIYQAGNKPCNGVAGFNCYFPLKSYSFRPTYGVGHQPYRV

SEQ ID NO: 9 - protein, 89 aa, synthetic construct
VVLSFELLHAPATVCGPKKSTNLVKNKCVNNFNGLKGTGVLTESNKKFLPFQQFGRDIADTTDAVRDPQTLEILDITPCSFGGVSVITP

SEQ ID NO: 10 - protein, 110 aa, synthetic construct
GTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNN

SEQ ID NO: 11 - protein, 100 aa, synthetic construct
SIAIPNNFTISVTTEILPVSMTKTSVDCTMYICGDSTECINLLLQYGSFCTQLKRALTGIAVEQDKNTQEVFAQVKQIYKTPPIKYFGGFNFSQILPDPS

SEQ ID NO: 12 - protein, 101 aa, synthetic construct
KPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGV

SEQ ID NO: 13 - protein, 79 aa, synthetic construct
TQNVLYENQKLIANQFNSAIGKIQDSLSSTASALGKLQDVVNHNAQALNTLVKQLSSKFGAISSVLNDIFSRLDKVEAE

SEQ ID NO: 14 - protein, 80 aa, synthetic construct
VQIDRLITGRLQSLQTYVTQQLIRAAEIRASANLAATKMSECVLGQSKRVDFCGKGYHLMSFPQSAPHGVVFLHVTYVPA

SEQ ID NO: 15 - protein, 40 aa, synthetic construct
QEKNFTTAPAICHDGKAHFPREGVFVSNGTHWFVTQRNFY

SEQ ID NO: 16 - protein, 37 aa, synthetic construct
NNATNVVIKVCEFQFCNDPFLDHKNNKSWMESEFRVY

SEQ ID NO: 17 - protein, 60 aa, synthetic construct
EIYQAGNKPCNGVAGFNCYFPLKSYSFRPTYGVGHQPYRVVVLSFELLHAPATVCGPKKS

SEQ ID NO: 18 - protein, 96 aa, synthetic construct; identical to SEQ ID NO: 7

SEQ ID NO: 19 - protein, 40 aa, synthetic construct
VVNHNAQALNTLVKQLSSKFGAISSVLNDIFSRLDKVEAE

SEQ ID NO: 20 - protein, 90 aa, synthetic construct
GTNTSNQVAVLYQGVNCTEVPVAIHADQLTPTWRVYSTGSNVFQTRAGCLIGAEYVNNSYECDIPIGAGICASYQTQTKSHRRARSVASQ

(19) United States
(12) Patent Application Publication
(10) Pub. No.: US 2024/0131139 A1
(43) Pub. Date: Apr. 25, 2024

(54) ADJUVANTED SUBCUTANEOUSLY-ADMINISTERED POLYPEPTIDE SARS-COV-2 VACCINES COMPOSITION AND METHODS

(71) Applicant: D4 Labs, LLC, Pacific Grove, CA (US)

(72) Inventors: Michael Bowe, Marina, CA (US); David Craig Wright, Pacific Grove, CA (US)

(21) Appl. No.: 17/983,659

(22) Filed: Nov. 9, 2022

Related U.S. Application Data
(60) Provisional application No. 63/419,101, filed on Oct. 25, 2022.

(51) Int. Cl. A61K 39/12; A61K 9/127

(52) U.S. Cl. CPC A61K 39/12; A61K 9/1272; A61K 9/1277; A61K 2039/55555

(57) ABSTRACT

Disclosed herein are subcutaneously administered, immunogenic compositions (e.g., vaccines) and methods of using and preparing the same. In some embodiments, the immunogenic compositions generate IgG antibodies to the spike proteins of the Wuhan-Hu-1, Delta B.1.617.2, and Omicron BA.1 variants of SARS-CoV-2 and may be suitable for use in preventing an infectious disease, such as SARS-CoV-2.

### CROSS-REFERENCE TO RELATED APPLICATION

This application claims priority to U.S. Provisional Patent Application Ser. No. 63/419,101, filed Oct. 25, 2022, the contents of which are incorporated by reference herein.

### SEQUENCE LISTING

The instant application contains a Sequence Listing which has been submitted electronically in ST.26 XML format and is hereby incorporated by reference in its entirety. Said ST.26 XML file, created on Mar. 20, 2023, is named 31579105002 PCT_Sequence_Listing.xml and is 22.8 kilobytes in size.

### FIELD OF THE INVENTION

Disclosed herein are features of subcutaneously-administered, adjuvanted polypeptide vaccines that generate antibody responses that persist at least until day 120 after vaccination, and methods for preparing and using the same. In particular, adjuvanted subcutaneously-administered SARS-CoV-2 polypeptide vaccines are disclosed. Advantageously, the adjuvanted subcutaneously-administered polypeptide vaccines disclosed herein do not utilize squalene.

### BACKGROUND OF THE INVENTION

Vaccine development and usage over the years has significantly reduced the number of infections and diseases on a global basis. The need for vaccines persists, however, including for the treatment of emerging viral threats (e.g., SARS-CoV-2) and other viral agents.

Vaccines are based on the use of an intact viral or bacterial agent, either inactivated or live attenuated, cloned expressed proteins using molecular biology techniques, or mRNA.

Three of the vaccines approved for use in the United States are either mRNA-based vaccines directed against the spike protein or adjuvanted baculovirus-expressed spike protein-based vaccines. Recently, SARS-CoV-2 vaccines have been approved that utilize just the receptor binding domain (RBD) of the spike protein, rather than the entire spike protein (Dai et al).

These approved vaccines frequently require two or more injections intramuscularly to develop a significant immune response. Additional boosters are necessary to sustain immunity. Further, administration of standard vaccines intramuscularly results in significant pain and swelling at the injection site. Other more serious reactions may occur.

There remains a need for novel vaccine strategies, particularly for emerging and recalcitrant viral diseases.

### SUMMARY OF THE INVENTION

Disclosed herein are features of adjuvanted polypeptide vaccines, and methods for preparing and using the same.

In a first aspect, an adjuvanted penta-polypeptide vaccine is disclosed comprising five synthetic peptides and one liposome, wherein the liposome is a non-phospholipid liposome incorporating Vitamin E, and wherein at least five synthetic polypeptides are encapsulated within the liposome.

In one embodiment, the adjuvanted penta-polypeptide vaccine disclosed herein comprises five linear peptides. In certain embodiments, the adjuvanted penta-polypeptide vaccine generates antibodies that recognize full-length spike proteins from the Wuhan variant, the Delta variant, and the Omicron BA. 1 variant of SARS-CoV-2.

In a particular embodiment, five synthetic peptides are synthesized from a known viral protein sequence, and more particularly the spike protein of SARS-CoV-2.

In one embodiment, the adjuvanted peptide vaccine comprises five linear peptides derived from SEQ ID NO: 2 (modified spike protein of SARS-CoV-2 Omicron BA. 1 variant) or a variant or homolog thereof, for example, a variant comprising one or more substitution mutations.

In certain embodiments, the adjuvanted peptide vaccine comprises five linear peptides comprising amino acids 121-157 (SEQ ID NO: 16), 305-400 (SEQ ID NO:17), 471-530 (SEQ ID NO: 18), 601-690 (SEQ ID NO: 19), and 951-990 (SEQ ID NO: 20) of SEQ ID NO: 2, or variants or homologs thereof, e.g., a variant comprising one or more substitution mutations.

In a particular embodiment, the adjuvanted peptide vaccine comprises five or more linear sequences selected from the group consisting of 12 constructs (SEQ ID NOS: 4-15).

In one embodiment, the liposome comprises a lipid bilayer comprising one or more non-ionic surfactants and optionally, a helper lipid (e.g., cholesterol).

In a particular embodiment, the lipid bilayer comprises between two (2) and ten (10) bilayers surrounding an amorphous central cavity. In certain embodiments, the lipid bilayer incorporates Vitamin E. In one embodiment, the central cavity of the liposome comprises Vitamin E.

In a second aspect, a method is disclosed for generating an immune response comprising administering the adjuvanted penta-polypeptide vaccine disclosed herein to a subject, thereby generating an IgG immune response.

In a third aspect, a method is disclosed for preventing an infection in a subject in need thereof comprising subcutaneously administering the adjuvanted penta-polypeptide vaccine disclosed herein to the subject, thereby preventing the infection, i.e., conferring protective immunity.

In certain embodiments, the adjuvanted penta-polypeptide vaccines disclosed herein increase the immune response to SARS-CoV-2 spike protein.

In certain embodiments, the adjuvanted penta-polypeptide vaccine is administered in one or more doses.

In certain embodiments, the adjuvanted penta-polypeptide vaccine is administered subcutaneously.

### BRIEF DESCRIPTION OF THE FIGURES

FIG. 1: Depicts blot images for IgG antibodies directed against the full-length spike protein of the 2019 Wuhan SARS-CoV-2 sequence. The animals were immunized subcutaneously on days 1 and 28 with the Omicron BA. 1 penta-polypeptide encapsulated in the SVE72 nonphospholipid-based liposome. Positive blots are red dots. The dilution of the sera was 1:500. Five out of five animals immunized with an adjuvanted penta-polypeptide vaccine are positive for IgG antibodies directed against the 2019 Wuhan SARS-CoV-2 spike protein at a 1:500 dilution of serum, 21 days and 71 days after the second dose of vaccine.

FIG. 2. Depicts blot images for IgG antibodies directed against the full-length spike protein of the SARS-CoV-2 Delta variant sequence. The animals were immunized subcutaneously on days 1 and 28 with the Omicron BA. 1 penta-polypeptide encapsulated in the SVE72 nonphospholipid-based liposome. Positive blots are red dots. The dilution of the sera was 1:500. Five out of five animals immunized with an adjuvanted penta-polypeptide vaccine are positive for IgG antibodies directed against the SARS-CoV-2 Delta variant spike protein at a 1:500 dilution of serum, 21 days and 71 days after the second dose of vaccine.

FIG. 3. Depicts blot images for IgG antibodies directed against the full-length spike protein of the SARS-CoV-2 Omicron BA. 1 variant sequence. The animals were immunized subcutaneously on days 1 and 28 with the Omicron BA. 1 penta-polypeptide encapsulated in the SVE72 nonphospholipid-based liposome. Positive blots are red dots. The dilution of the sera was 1:500. Five out of five animals immunized with an adjuvanted penta-polypeptide vaccine are positive for IgG antibodies directed against the SARS-CoV-2 Omicron BA. 1 variant spike protein at a 1:500 dilution of serum, 21 days and 71 days after the second dose of vaccine.

FIG. 4. Depicts blot images for IgG antibodies directed against the full-length spike protein of the SARS-CoV-2 Omicron BA. 1 variant sequence. The animals were immunized subcutaneously on days 1 and 28 with the Omicron BA. 1 penta-polypeptide encapsulated in the SVE72 nonphospholipid-based liposome. Positive blots are red dots. The dilution of the sera was from 1:500 to 1:4000. Five out of five animals immunized with an adjuvanted penta-polypeptide vaccine are positive for IgG antibodies directed against the SARS-CoV-2 Omicron BA.1 variant spike protein at a 1:2000 dilution of serum, at day 120, which is 71 days after the second dose of vaccine.

FIG. 5. Depicts particle sizing results for vaccines SVE52.O.DCS.37.139 from two lots of vaccine production corresponding to the immunization on DS1 (Lot #021522) and the immunization on DS28 (Lot #031522). Sizing was done on the Beckman Coulter Laser Sizer LS 13 320 XR. Lot #021522 sizing was done on 02-22-22 and eight months later, on 10-04-22. Lot #031522 sizing was done on 03-15-22 and seven months later, on 10-07-22. 200 μL of sample were diluted into 10.0 mL dH2O, loaded onto the device until the target quantity reached 6-8% and the size was measured. Data is reported as an average of two runs in nm and the percent change reported shows product stability with a change in size of <10%.

FIG. 6. Depicts particle sizing results for vaccines SVE72.O.DCS.37.139 from two lots of vaccine production corresponding to the immunization on DS1 (Lot #021522) and the immunization on DS28 (Lot #031522). Sizing was done on the Beckman Coulter Laser Sizer LS 13 320 XR. Lot #021522 sizing was done on 02-22-22 and eight months later, on 10-04-22. Lot #031522 sizing was done on 03-15-22 and seven months later, on 10-07-22. 200 μL of sample were diluted into 10.0 mL dH2O, loaded onto the device until the target quantity reached 6-8% and the size was measured. Data is reported as an average of two runs in nm and the percent change reported shows product stability with a change in size of <10%.

Sequence 1. Depicts the surface glycoprotein of Severe acute respiratory syndrome coronavirus 2, accession number UG097992.1 from the NCBI protein database.

Sequence 2. Depicts SARS-CoV-2 mutations from variant B.1.1.529 Omicron (71 isolates in South Africa collected Nov. 11, 2021) superimposed on the surface glycoprotein [SARS-CoV-2, China, Human Isolate, 2/18/20]: QJG65958.1.

Sequence 3. Depicts a modification of Sequence 2.

Sequence 4-15. Depicts exemplary embodiments of twelve polypeptides derived from Sequence 3.

Sequence 16-20. Depicts the five polypeptides selected from SEQ ID NOS: 4-15 for use in the current vaccine described.

### DETAILED DESCRIPTION

### I. Definitions

The term “about” as used herein refers to a value or element that is similar to a stated reference value or element. In certain embodiments, the term “about” or “approximately” refers to a range of values or elements that falls within 25%, 20%, 19%, 18%, 17%, 16%, 15% 14% 13% 12% 11% 10% 9% 8% 7% 6% 5% 4% 3% 2% 1%, or less in either direction (greater than or less than) of the stated reference value or element unless otherwise stated or otherwise evident from the context (except where such number would exceed 100% of a possible value or element).

The term “adjuvant” as used herein refers to a substance whose admixture with an administered immunogenic determinant/antigen construct increases or otherwise modifies the immune response to said determinant. Immunological adjuvants function by attracting macrophages to the antigen and then to presenting said antigens to the regional lymph nodes and initiating an effective antigenic response. Conventional adjuvants can serve as vehicles for the antigen, and as nonspecific immunological stimulants. In one embodiment, the liposome (e.g., the paucilamellar liposome) serves as an adjuvant for the polypeptide (e.g., penta-polypeptide) vaccine disclosed herein and in certain embodiments, incorporates Vitamin E.

The term “administering” as used herein means either directly administering a compound or composition of the present invention. Any route of administration, such as topical, subcutaneous, peritoneal, intravenous, intraarterial, inhalation, vaginal, rectal, nasal, buccal, introduction into the cerebrospinal fluid, or instillation into body compartments can be used. The terms and phrases “administering” and “administration of,” when used in connection with a compound or pharmaceutical composition (and grammatical equivalents) refer both to direct administration, which may be administration to a patient by a medical professional or by self-administration by the patient, and/or to indirect administration, which may be the act of prescribing a drug.

The term “affinity” as used herein refers to the equilibrium constant for the reversible binding of two agents and is expressed as a dissociation constant (K_{D}).

The term “amino acid” or “amino acids” as used herein is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids (stereoisomers). Abbreviations for amino acids are well understood in the art.

The terms “amino-terminal” and “carboxyl-terminal” are used herein to denote positions within polypeptides. Where the context allows, these terms are used with reference to a particular sequence or portion of a peptide to denote proximity or relative position.

The term “amphiphilic” as used herein means exhibiting characteristics of hydrophilicity and lipophilicity. Common amphiphilic substances are soaps, detergents and lipoproteins. Other examples of amphiphilic compounds are: saponins, phospholipids, glycolipids, polysorbates.

The term “antigen” as used herein refers to a molecule with one or more epitopes that stimulate a host's immune system to make a secretory, humoral and/or cellular antigen-specific response, or to a DNA molecule that is capable of producing such an antigen in a vertebrate. The term is also used interchangeably with “immunogen.” For example, a specific antigen can be complete protein, portions of a protein, peptides, fusion proteins, glycosylated proteins and combinations thereof.

The term “binding” as used herein refers to direct association between two molecules, due to, for example, covalent, electrostatic, hydrophobic, and ionic and/or hydrogen-bond interactions, including interactions such as salt bridges and water bridges. “Specific binding” refers to binding with an affinity of at least about 10⁻⁷ M or greater.

The term “boost” as used herein refers to the administration of an additional dose of an immunizing agent, such as a vaccine, administered at a time after the initial dose to sustain the immune response elicited by the previous dose of the same agent.

The term “carrier” as used herein includes any solvent(s), dispersion medium, coating(s), diluent(s), buffer(s), isotonic agent(s), solution(s), suspension(s), colloid(s), inert (s), or such like, or a combination thereof that is pharmaceutically acceptable for administration to the relevant animal or acceptable for a therapeutic or diagnostic purpose, as applicable.

The terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The term “conservative amino acid substitution” as used herein refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

The term “cross-reacts” as used herein refers to the reaction between an antigen and an antibody that was generated against a different but similar antigen.

The term “dodecapolypeptide” as used herein refers to twelve unique polypeptides in the same liposome preparation.

The term “encapsulate” as used herein refers to the lipid vesicle forming an impediment to free diffusion into solution by an association with or around an agent of interest, e.g., a lipid vesicle may encapsulate an agent within a lipid layer or within an aqueous compartment inside or between lipid layers.

The term “identical” or percent “identity,” in the context of two or more nucleic acids or polypeptide sequences, may refer to two or more sequences or subsequences that are the same or have a specified percentage of amino acid residues or nucleotides that are the same. In some cases, 2 or more sequences may be homologous (homologs) if they share at least 20% 25% 30%. 35% 40% 45% 50% 55% 60% 65% 70% 75% 80% 85% 90% 91% 92%, 93%, 94%, 95%, 96%, 97%, 98%, 99%, or higher identity to a reference sequence when compared and aligned for maximum correspondence over a comparison window, or designated region as measured using one of the following sequence comparison algorithms or by manual alignment and visual inspection. In some cases, 2 or more sequences 2 or more sequences may be homologous if they share at most 20%, 25%, 30%. 35%, 40%, 45% 50%, 55%, 60%, 65%, 70%, 75%, 80%, 85%, 90%, 91%, 92%, 93%, 94%, 95%, 96%, 97%, 98%, 99% or higher identity to a reference sequence. This definition also refers to the compliment of a test sequence. Preferably, the identity exists over a region that is at least 25 amino acids or nucleotides in length or in some cases over a region that is 50-100 amino acids or nucleotides in length. In some cases, 2 or more sequences may be homologous and share at least 30% identity over at least 80 amino acids in a sequence according to the Sander-Schneider homology limit.

The term “incorporating” or “incorporated” as used herein with reference to a liposome means encapsulated/encapsulating into the cavity of the liposome, within the potential double layer of the liposome, or as part of the membrane layer of the liposome.

The term “immune response” as used herein refers to a response of a cell of the immune system, such as a B cell, T cell, dendritic cell, macrophage or polymorphonucleocyte, to a stimulus such as an antigen or vaccine. An immune response can include any cell of the body involved in a host defense response, including for example, an epithelial cell that secretes an interferon or a cytokine. An immune response includes, but is not limited to, an innate and/or adaptive immune response. As used herein, a protective immune response refers to an immune response that protects a subject from infection (e.g., prevents infection or prevents the development of disease associated with infection). Methods of measuring immune responses are well known in the art and include, for example, by measuring proliferation and/or activity of lymphocytes (such as B or T cells), secretion of cytokines or chemokines, inflammation, antibody production and the like. “Enhancing an immune response” refers to co-administration of an adjuvant and at least one peptide, wherein the adjuvant increases the desired immune response to the at least one peptide compared to administration of the at least one peptide in the absence of the adjuvant.

The term “immunogenic composition” as used herein are those which result in specific antibody production or in cellular immunity when injected into a subject. In certain embodiments, the vaccine disclosed elicits a neutralizing antibody response.

The term “immunogenic variants” as used herein, refers to a variant that that is predicted to be immunogenic.

The term “linker” as used herein refers to a molecule positioned between two moieties. Typically, linkers are bifunctional, i.e., the linker includes a functional group at each end, wherein the functional groups are used to couple the linker to the two moieties.

The term “liposome” as used herein refers to a vesicle made of concentric bilayers of lipids and more particularly, non-phospholipids. The liposome can be formed of the same lipid or different lipids. These lipids can have an anionic, cationic or zwitterionic hydrophilic head group. The size of a liposome may vary but is generally from about 10 to about 3000 nm. In certain embodiments, the liposome has an aqueous core, while in other embodiments, the liposome has an oil and water-filled core. The term “empty liposome” as used herein refers to a liposome not incorporating any peptide or other antigen within the liposome core.

The term “multimellar” as used here refers to a vesicle containing more than one lipid bilayers.

The term “multimeric” as used herein with reference to a peptide antigen refers to a structure consisting of several peptides. If all the subunits are the same, these are called homomeric peptide. Homomeric proteins consist of the same kind of subunits that are held together by noncovalent bonds to form a bigger, whole structure (i.e. quaternary structure). the subunits are different, these are called heteromeric proteins

The term “non-ionic surfactant” as used herein refers to a class of surfactants which have no charge groups in their hydrophilic heads. In solutions, nonionic surfactants form structures in which hydrophilic heads are opposite to aqueous solutions and hydrophilic tails are opposite to organic solutions. Representative, non-limiting non-ionic surfactants include alkyl esters, alkyl amides, alkyl ethers and esters of fatty acids.

The term “paucilamellar” as used herein refers to a vesicle having 2-10 lipid bilayers. In certain embodiments disclosed herein the vesicle that contains five or more polypeptides is paucilamellar.

The term “peptide” as used herein refers to a sequence of three (2) or more amino acids and typically less than fifty (120) amino acids, wherein the amino acids are naturally occurring or non-naturally occurring amino acids. Non-naturally occurring amino acids refer to amino acids that do not naturally occur in vivo but which, nevertheless, can be incorporated into the peptide structures described herein. The peptide is preferably synthesized but may also be made recombinantly, for example. In some embodiments, a peptide can be between 2 and 10, about 8 and 40, about 12 and 60 or about 20 and about 80 amino acids in length. In certain embodiments, the peptide is 2, 3, 4, 5, 6, 7, 8, 9, 10, 11 or 12 amino acids in length.

The term “pharmaceutical composition” refers to a mixture of one or more chemicals, or pharmaceutically acceptable salts thereof, with a suitable carrier, for administration to a mammal as a medicine.

The term “phospholipid” as used herein refers to any of a group of lipids whose molecule has a hydrophilic “head” containing a phosphate group, and two hydrophobic “tails” derived from fatty acids, joined by a glycerol molecule. The phosphate group can be modified with simple organic molecules such as choline, ethanolamine or serine. In certain embodiments herein, the liposome does not contain phospholipids.

The terms “polypeptide” and “protein” are terms that are used interchangeably to refer to a polymer of amino acids, without regard to the length of the polymer. Typically, polypeptides and proteins have a polymer length that is greater than that of “peptides.” A penta-polypeptide is five unique polypeptides.

The term “prophylactic” as used herein with reference to an immunogenic composition (e.g., a vaccine) that is administered to a subject who does not exhibit signs of a disease.

The term “protein” as used herein refers to a sequence of amino acid residues more than 120 amino acids in length.

The term “receptor binding domain” as used herein refers to amino acids 319 to 541 in the S1 sequence of the spike protein of SARS-CoV-2.

The term “receptor binding motif” as used herein refers to amino acids 438 to 508 in the S1 sequence of the spike protein of SARS-CoV-2.

The term “recombinant” as used herein refers to intended to refer to peptides, polypeptides or proteins that are designed, engineered, prepared, expressed, created, or isolated by recombinant means, such as polypeptides expressed using a recombinant expression vector transfected into a host cell, polypeptides isolated from a recombinant, combinatorial polypeptide library, or polypeptides prepared, expressed, created or isolated by any other means that involves splicing selected sequence elements to one another. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source. In some embodiments, one or more such selected sequence elements results from the combination of multiple (e.g., two or more) known sequence elements that are not naturally present in the same polypeptide.

The term “spike protein”, as used herein, refers to a type I transmembrane glycoprotein that is characteristic of coronaviruses. Most spike proteins contain a leader, an ectodomain, a transmembrane domain and an intracellular tail.

The term “subject in need thereof” as used herein refers to a living organism suffering from or prone to a disease or condition that can be treated by using the methods provided herein. The term does not necessarily indicate that the subject has been diagnosed with a particular disease or disorder, but typically refers to an individual under medical supervision. Non-limiting examples include humans, other mammals, bovines, rats, mice, dogs, monkeys, goat, sheep, cows, deer, and other non-mammalian animals. In embodiments, a patient is human.

The term “substitution” as used herein with reference to a peptide refers to the replacement of one amino acid residue by a different amino acid residue. In certain embodiments, the substitution is conservative. Conservative amino acid substitutions include: (i) small aliphatic, nonpolar or slightly polar residues: Ala, Ser, Thr, Pro, Gly; (ii) polar, negatively charged residues and their amides and esters: Asp, Asn, Glu, Gln, cysteic acid and homocysteic acid; (iii) polar, positively charged residues: His, Arg, Lys; Ornithine (Om); (iv) large, aliphatic, nonpolar residues: Met, Leu, Ile, Val, Cys, Norleucine (Nle), homocysteine; and (iv) large, aromatic residues: Phe, Tyr, Trp, acetyl phenylalanine

The term “penta-polypeptide” as used herein refers to five unique polypeptides in the same liposome preparation.

The term “therapeutically effective amount” as used herein refers an amount sufficient to prevent, correct and/or normalize an abnormal physiological response. In one aspect, a “therapeutically effective amount” is an amount sufficient to reduce by at least about 30 percent, more preferably by at least 50 percent, most preferably by at least 90 percent, a clinically significant feature of pathology, such as for example, size of a tumor mass.

The term “therapeutic activity” or “activity” may refer to an activity whose effect is consistent with a desirable therapeutic outcome in humans, or to desired effects in non-human mammals or in other species or organisms. Therapeutic activity may be measured in vivo or in vitro. For example, a desirable effect may be assayed in cell culture.

The terms “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance.

The term “vaccine” as used herein, refers to any type of biological preparation contributing to or soliciting active immune responses against a particular disease or pathogen. Such biological preparation can include, but is not limited to, an antigen derived from a disease-causing agent or a portion of an antigen derived from a disease-causing agent.

The term “vaccination” or “vaccinate” refers to the administration of a composition intended to generate an immune response, for example to a disease-causing agent.

Vaccination can be administered before, during, and/or after exposure to a disease-causing agent, and/or to the development of one or more symptoms, and in some embodiments, before, during, and/or shortly after exposure to the agent. In some embodiments, vaccination includes multiple administrations, appropriately spaced in time, of a vaccinating composition.

The term “vaccine efficacy” or “VE” as used herein measure the proportionate reduction in cases among vaccinated persons. It is measured by calculating the risk of disease among vaccinated and unvaccinated persons and determining the percentage reduction in risk of disease among vaccinated persons relative to unvaccinated persons. The greater the percentage reduction of illness in the vaccinated group, the greater the vaccine efficacy.

The term “variant” as used refers to a polynucleotide sequence related to a wild type gene. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence. A “variant” of a protein or peptide may have at least 70%, 75%, 80%, 85%, 90%, 95%, 98% or 99% amino acid identity over a stretch of 10, 20, 30, 50, 75 or 100 amino acids of such protein or peptide. Variants of the proteins or peptides as defined herein, which may be encoded by a nucleic acid molecule, may also comprise those sequences, wherein nucleotides of the encoding nucleic acid sequence are exchanged according to the degeneration of the genetic code, without leading to an alteration of the respective amino acid sequence of the protein or peptide, i.e. the amino acid sequence or at least part thereof may not differ from the original sequence in one or more mutation(s) within the above meaning.

The term “vesicle” as used herein refers to a structure consisting of liquid or cytoplasm enclosed by a lipid bilayer. The interior of the vesicle is typically an aqueous environment but can also be an oily environment, and it may comprise an agent such as but not limited to a prophylactic, therapeutic or diagnostic agent.

### II. Immunogenic Compositions

The present disclosure provides immunogenic compositions (e.g., vaccines) and pharmaceutical compositions comprising at least one polypeptides (e.g., a synthetic polypeptide) and a liposome (e.g., a non-phospholipid liposome incorporating Vitamin E). In certain embodiments, at least one polypeptides are encapsulated within the liposome. These compositions are suitable for use, for example, in generating an immune response as described further herein.

In certain embodiments, the immunogenic composition comprises at least two, at least three, or at least four or at least five or more polypeptides.

In a particular embodiment, the immunogenic composition comprises five polypeptides.

The peptides may be linear or multimeric.

A. Polypeptides

The immunogenic compositions (e.g., vaccines) disclosed herein include at least on (e.g., five) polypeptides, such as synthetic immunogenic polypeptides. In certain embodiments, the immunogenic compositions contain a combination of polypeptides. In certain embodiments, the immunogenic composition cross-reacts with full-length SARS-CoV-2 spike proteins.

In one embodiment, the immunogenic composition comprises at least one polypeptide (e.g., five) that contains a portion of the receptor binding motif of the Omicron BA. 1 variant and a purported T cell epitope. Shown in SEQ ID NOS: 16-20(CS18007 D-802 DCS.O.60) are the sequence of the full-length receptor binding motif (RBM) of Omicron BA. 1 and the portion of the RBM with a T cell epitope in the C terminus portion of the polypeptide. According to this embodiment, the peptide sequence has 33 fewer amino acids on the N terminus end of the RBM and 22 additional amino acids containing a purported T cell epitope on the C terminus end of the polypeptide.

In one embodiment, the immunogenic compositions comprises a polypeptide selected from the group of SEQ ID NOS: 4-15 or a combination thereof.

In one embodiment, the one or more polypeptides are linear polypeptides. In certain embodiments, the immunogenic composition comprises at least five synthetic immunogenic linear polypeptides, where all synthetic immunogenic linear polypeptides are different (i.e., mixed).

In a particular embodiment, the immunogenic composition comprises at least one (e.g., five) polypeptides derived from viral proteins, and more particularly viral proteins of SARS-CoV-2.

In a particular embodiment, the immunogenic composition comprises at least one (e.g., five) polypeptides derived from the spike (S) protein of SARS-CoV-2. At least one polypeptide may be a polypeptide that is currently known or unknown, i.e., occurs currently or represents a predicted mutation.

In one embodiment, the immunogenic composition comprises at least five polypeptides of the twelve polypeptides identified, shown in SEQ ID NOS: 4-15.

In one embodiment, the immunogenic composition comprises two or more (e.g., five) linear peptides derived from SEQ ID. NO: 2 or a variant or homolog thereof, for example, a variant or homolog comprising one or more substitutions.

The synthetic polypeptide may be any suitable synthetic polypeptide or antigen. The synthetic peptide may be derived, for example, from an infectious agent selected from a virus, bacteria, fungi, parasite, or phon.

In one embodiment, at least one synthetic polypeptide is derived from viral peptide or antigen. Virion particles are generally comprised of genetic material (e.g., DNA or RNA), a protein coat, and a lipid envelope and use receptors and co-receptors to enter a cell.

In a particular embodiment, the at least one synthetic polypeptide is derived from a viral peptide from a double stranded DNA virus (dsDNA), a single stranded DNA virus (ssDNA), a double stranded RNA virus (dsRNA) or a single stranded RNA virus (ssRNA).

In one embodiment, the viral peptide is derived from a DNA virus selected from the group consisting of adenovirus, papillomavirus, parvovirus, herpes simplex virus, varicella-zoster virus, cytomegalovirus, Epstein-Barr virus, smallpox virus, vaccinia virus, and hepatitis B virus.

In another embodiment the viral peptide derived from RNA virus selected from the group consisting of tavirus, norovirus, enterovirus, hepatovirus, rubella virus, influenza viruses (A, B, and C), measles virus, mumps virus, hepatitis C virus, yellow fever virus, hantavirus, Zika virus, California encephalitis virus, rabies virus, Ebola virus, and HIV.

ii. Coronavirus

In one embodiment, the viral polypeptides are from a coronavirus. The coronavirus can be any coronavirus currently known, or later discovered. In certain embodiments, the coronavirus is zoogenic.

Coronaviruses are positive strand RNA viruses with the largest viral genome among the RNA viruses (27-33 kb). The virus particles are enveloped and carry extended spike proteins on the membrane surface, providing the typical crown-like structure. The coronaviruses share a conserved organization of their (positive strand) RNA genome. The 5′ two-thirds of the genome contains the large 1a and 1b ORFs, encoding the proteins necessary for RNA replication (the nonstructural proteins), whereas the 3′ one-third contains the genes coding for the structural proteins: haemagglutinin esterase protein (only for group IIa), spike protein, envelope protein, membrane protein and nucleocapsid protein. The accessory protein genes are interspersed between the structural protein genes and differ in number and position for the various coronavirus.

Several coronavirus genera and subgenera are recognized hips:/talk.ictvonline.org/ictv-reports/). Among them, alpha- and betacoronaviruses infect mammals, gammacoronaviruses infect avian species, and deltacoronaviruses infect both mammalian and avian species. The coronavirus may be, for example, 229E, SARS, MERS, SARS-CoV-1 (OC43), and SARS-CoV-2.

The coronavirus spike protein includes three segments: a large ectodomain, a single-pass transmembrane anchor, and a short intracellular tail The ectodomain consists of a receptor-binding subunit S1 and a membrane-fusion subunit S2. The S1 and S2 domains may be separated by a cleavage site that is recognized by furin-like proteases during S protein biogenesis in the infected cell. The spike protein binds to a receptor on the host cell surface through the S1 subunit and then fuses viral and host membranes through its S2 subunit. The spike protein exists in two structurally distinct conformations, pre-fusion and post-fusion.

The S1 subunit of the betacoronavirus spike proteins displays a multidomain architecture and is structurally organized in four distinct domains A-D of which domains A and B may serve as a Receptor Binding Domain (RBD).

In a particular embodiment, the viral polypeptides are from SARS-CoV-2 or a variant thereof. SARS-CoV-2 can cause severe respiratory illness and significant mortality among those over 65 years old or with chronic conditions. Infection of target cells by SARS-CoV-2 is mediated through the interaction of the viral Spike (S) protein (1273 amino acids or fewer) and its cellular receptor, angiotensin-converting enzyme 2 (ACE2). The SARS-CoV-2 receptor-binding domain in Sequence ID 3 (amino acids 319-541) includes a region along its periphery that contacts ACE2 and is designated the receptor-binding motif (in Sequence ID 3 amino acids 438-508).

In one embodiment, the viral polypeptides are derived from the spike (S) protein of the coronavirus. Other relevant proteins like envelope (E), membrane (M), nucleocapsid (N) protein of the coronavirus or a combination thereof could occur.

In one embodiment, the vaccine contains at least five synthetic polypeptides derived from the spike protein (amino acid numbers 1-1,110).

In one embodiment, at least one synthetic polypeptide is derived from a fragment of the S1 or S2 domains. The fragment of the S1 or S2 domains may include 30, 40, 50, 60, 90, or 100 amino acids of the S1 or S2 domain.

In certain embodiments, the vaccine contains at least one (e.g., five) synthetic polypeptide is/are derived from the Receptor Binding Domain (RBD) (amino acid number 319-541) of the spike protein.

In certain embodiments, the vaccine contains at least one polypeptide (e.g., five) derived from the RBD of SEQ. ID NO: 2, which is the SARS-CoV-2 Omicron BA.1 Spike Protein sequence from Utah Public Health Laboratory collected on 1-20-2022 (Accession #URD15893.1) from the National Center for Biotechnology Information protein database.

In one embodiment, the vaccine contains at least five polypeptides selected from the group of twelve polypeptides shown in SEQ ID NOS: 4-15.

In another embodiment, the two peptides are derived from SEQ ID NO: 2 having one or more mutations, e.g., a substitution, insertion, deletion or inversion. In a particular embodiment, the variant sequence has at least 80%, 85%, 90%, 93%, 95%, 96%, 97%, 98%, 99%, 99.5%, 99.6%, 99.7%, 99.8%, 99.9%, 99.95%, 99.98% or 99.99% sequence identity to NO: 2.

B. Lipid Vesicle

In some embodiments, at least one polypeptide (e.g., synthetic polypeptide) is provided with (e.g., encapsulated within) a lipid vesicle.

Lipid vesicles are substantially spherical structures made of amphiphiles, e.g., surfactants or phospholipids. The lipids of these spherical vesicles are generally organized in the form of lipid bilayers, e.g., multiple onion-like shells of lipid bilayers which encompass an aqueous volume between the bilayers. Certain types of lipid vesicles have an unstructured central cavity which can be used to encapsulate and transport a variety of materials. The lipid vesicle may be charged or neutral.

The lipid vesicle may be any suitable lipid vesicle such as a liposome, e.g., a non-phospholipid-based liposome comprising an adjuvanting oil. The lipid vesicle may be a unilamellar or multilamellar vesicle. Multilamellar vesicles are concentric circles constructed by at least 2 bilayer vesicles or a large vesicle embodying one or more small vesicles.

In one embodiment, the lipid vesicle is a liposome.

Liposome properties differ and may be selected on the basis of lipid composition, surface charge, size, and the method of preparation. Typically, liposomes can be divided into three categories based on their overall size and the nature of the lamellar structure. In one embodiment, the liposome is a small unilamellar vesicle (SUV) between about 20 and about 100 nm, a large unilamellar vesicle (LUV) greater than about 100 nm, a giant unilamellar vesicle (GULV) greater than about 100 nm, an oligolamellar vesicle (OLV) between about 100 and about 1000 nm or a multilamellar large vesicle (MLV) greater than about 500 nm.

In one embodiment, the vesicle is a liposome. The liposome may be formed of one or more non-phospholipic amphiphiles and more particularly, one or more non-ionic surfactants and optionally, a membrane stabilizer such as cholesterol. Membrane stabilizers may be included to improve one or more properties of the liposome.

The lipid vesicle is a non-phospholipid-based liposome containing vitamin E. The lipid vesicle may be a unilamellar or multilamellar vesicle. Multilamellar vesicles are concentric circles constructed by at least 2 bilayer vesicles or a large vesicle embodying one or more small vesicles.

In one embodiment, the lipid vesicle is a non-phospholipid-based liposome composed of polyoxyethelene 2-cetyl ether, cholesterol, oleic acid, vitamin E, and sterile water, containing the protein.

In one embodiment, the lipid vesicle is a non-phospholipid-based liposome composed of polyoxyethelene 2-stearyl ether, cholesterol, oleic acid, vitamin E, and sterile water, containing the protein.

In certain embodiments, the liposome further comprises polyoxyethylene fatty acid ethers (polyoxyethylene 2-stearyl or cetyl ethers), at least one sterol consisting of cholesterol as a membrane stabilizer, a negative charge producing agent (oleic acid), Vitamin E and any lipid soluble or water-soluble materials to be incorporated into the vesicles.

Liposome properties may differ and can be selected on the on the basis of lipid composition, surface charge, size, and the method of preparation.

In one embodiment, the lipid vesicle is a liposome selected from a small unilamellar vesicle (SUV) (10-100 nm), a large unilamellar vesicle (LUV) (100-3000 nm) and multi-lamellar vesicle (MLV). In certain embodiments, liposome is a vesicle comprising between 2 and about 10 layers. The 2 to 10 peripheral bilayers encapsulate an aqueous volume which is interspersed between the lipid bilayers and may also be encapsulated in the amorphous central cavity. Alternatively, the amorphous central cavity may be substantially filled with a water immiscible material, such as an oil or wax. The paucilamellar vesicles containing such amphiphiles provide a high carrying capacity for water-soluble and water immiscible substances. The high capacity for water immiscible substances represents a unique advantage over classical phospholipid multilamellar liposomes.

The lipid vesicle contains a central cavity, carrying either water soluble materials or a water-immiscible oily solution, which can be used to encapsulate the antigen. The water-immiscible oily solution is made of materials which are both water immiscible and immiscible in the lipids used to form the bilayers. The water immiscible oily material found in the amorphous central cavity is comprised of Vitamin E.

In certain embodiments, oleic acid can insert in the membrane allowing negatively charged structures to be produced.

C. Pharmaceutical Composition

In certain embodiments, the immunogenic composition (e.g., vaccine) includes a pharmaceutically acceptable carrier. The pharmaceutically acceptable carrier may be formulated for administration to a human subject or animal.

In some embodiments, the pharmaceutically acceptable carrier includes diluents and adjuvants. Diluents include, for example, water or saline.

Compositions may be prepared as injectables, either as liquid solutions or suspensions. The composition may be prepared for nasal administration, e.g. as drops. Compositions may be presented in vials, or they may be presented in ready-filled syringes. The syringes may be supplied with or without needles. A syringe will include a single dose of the composition, whereas a vial may include a single dose or multiple doses.

The composition may be packaged in unit dose form or in multiple dose form.

### III. Methods of Use

The immunogenic compositions and vaccines described herein are useful, for example, for generating an immune response. The method includes contacting the cell with an effective amount of the immunogenic composition described herein.

In some embodiments, methods of inducing an immune response are used for vaccination. The methods involve administering a prophylactically-effective amount of the immunogenic composition as described herein to prevent an infection by, or an amount sufficient to reduce the biological activity of, an infectious agent such as a virus (e.g., a coronavirus).

In certain embodiments, the vaccine is a prophylactic vaccine, i.e., confers immunity to a subject who is not infected. According to this embodiment, the method comprising administering the vaccine to a subject in need thereof. In certain embodiments, administration is subcutaneously.

For example, and without limitation, the one or more subsequent exposures occurring administration may result in reduced viral titers, reduced amount and/or severity of symptoms, shortened duration of symptoms, and/or reduced need for treatment medications and/or clinician oversight, as compared to a control.

In certain embodiments, the immunogenic composition (e.g., vaccine) is administered to a subject as a single dose followed by a second dose later. In one embodiment the immunogenic composition (e.g., vaccine) and/or booster administrations may be repeated and such administrations may be separated by at least 28 days after the initial dose.

In one embodiment, the vaccine disclosed herein is administered as a booster to one or more vaccines known in the art. In a particular embodiment, the vaccine disclosed herein is administered as a booster to an mRNA vaccine or an adenoviral vaccine. In a particular embodiment, the vaccine disclosed herein is a booster to a SARS-CoV-2 vaccine selected from the Pfizer-BioNTech COVID-19 vaccine, the Moderna COVID-19 vaccine, the Janssen COVID-19 vaccine, or the Novavax COVID-19 vaccine.

Administration may be by any suitable mode known in the art. In a particular embodiment, administration is subcutaneous.

The useful dosage administered may vary. In one embodiment, the suitable dose is about 70 μg of antigen.

Dosage can be by a single dose schedule or a multiple dose schedule. Multiple doses may be used in a primary immunization schedule and/or in a booster immunization schedule. In a multiple dose schedule the various doses may be given by the same or different routes, e.g., a parenteral prime and mucosal boost, a mucosal prime and parenteral boost, etc. Multiple doses will typically be administered at least about 3 weeks apart, more particularly about 4 weeks apart.

The subject may be an animal, preferably a vertebrate, more preferably a mammal. Exemplary subject includes, e.g., a human, a cow, a pig, a chicken, a cat or a dog, as the infectious agents covered herein may be problematic across a wide range of species. Where the vaccine is for prophylactic use, the human is preferably a teenager, or an adult.

### V. Methods of Preparation

The immunogenic compositions (e.g., vaccines) disclosed herein may be prepared by any suitable method.

The polypeptide of the immunogenic composition disclosed herein can be made in any suitable way. Polypeptides are chemically synthesized.

Once a lipophilic phase is made, it is blended with an aqueous phase (e.g., water, saline, or any other aqueous solution which will be used to hydrate the lipids), which contain polypeptide antigens, under shear mixing conditions to form the adjuvanted polypeptide vaccine.

In one embodiment, the lipid vesicle is prepared utilizing high sheer technology.

In one embodiment, the lipid vesicle (e.g., non-phospholipid-based liposome) is loaded by direct entrapment.

The final concentration of peptide in the adjuvanted vaccine may vary. In one embodiment, the final concentration is 70 μg of each peptide in each vaccine dose.

The following Examples will clearly illustrate the efficacy of the invention.

### EXAMPLES

### Example 1: Preparation of Peptides

A variety of peptides were prepared as described below.

A. Solid Phase Peptide Synthesis

In all cases the Solid Phase Peptide Synthesis (SPPS) was carried out using a CSBio II SPPS Instrument. All synthesis was conducted on Rink amide resin using standard Fmoc conditions. Rink amide resin was added to the reaction vessel, which was clamped in the SPPS instrument. HOBt and the relevant protected amino acids (see Table I for details) were added to glass AA reservoirs, which were loaded into the instrument in order, from the C-terminal amino acid to the N-terminal amino acid. Stock solutions of diisopropylcarbodiimide (DIC) (0.5 M) in DMF and piperidine (20%) in DMF were prepared and loaded into the instrument, solvent delivery was accomplished with 6 psi of N2. The resin was pre-swelled with DMF (3 washes over 10 mm), followed by all coupling steps (piperidinemediated Fmoc-deprotection followed by DIC-mediated amide coupling, all done at 60 deg. C (total time per step approx. 40 min.), and a final Fmoc-deprotection step of the N-terminus.

Once the synthesis was complete, the resin was collected on a filter funnel by vacuum filtration and washed with DCM (3×50 mL) to facilitate removal of residual DMF followed by MeOH (3×50 mL) to shrink the resin. The resin-bound protected polypeptide was then dried under vacuum and stored at ambient temperature.

B. Cleavage and Deprotection

A 20 mL scintillation vial was charged with a stir bar and resin-bound peptide was added. Separately, a cleavage cocktail was prepared by adding triflouroacetic acid (TFA) 95%, triisopropylsilane (TIS) 2.5%, and purified water 2.5% to a 15 ml, plastic tube (for peptides with cystienes approx. 2% ethanedithiol (EDT) was added) and mixed well. The cleavage cocktail was added all at once to the scintillation vial. The vial was loosely capped (to allow escape of CO2) and the mixture was stirred for 3-4 hours at ambient temperature. At this point, the solid resin byproducts were removed by vacuum filtration, and the filtered crude product precipitated in cold Et2O. The precipitate was spun down at approx. 3000 rpm, the ether removed by decanting and this process repeated two more times. The solid precipitate was dried under vacuum, ready for purification.

C. Purification

The peptides were purified by reverse phase HPLC. HPLC was performed on a CI 8 protein column using dilute aqueous TFA (0.1 TFA, 99.9% Milli-Q purified water v/v) and acetonitrile (ACN) as eluents. The solvent gradient increased from 0% ACN to 75% ACN over 35 min, then to 90% ACN over 5 min and held at 90% ACN for 5 min. The product fractions were frozen at −80° C. then lyophilized to form a white powder. The compounds were, characterized for purity by analytical HPLC and for identity by mass spectrometry.

TABLE 1

Fmoc-Arg(Pbf)-OH
Fmoc-Asn(Trt)-OH
Fmoc-Cys(Trt)-OH
Fmoc-Glu(OtBu)-OH
Fmoc-Gln(Trt)-OH
Fmoc-Gly-OH
Fmoc-His(Trt)-OH
Fmoc-Leu-OH
Fmoc-Lys(Boc)-OH
Fmoc-Phe-OH
Fmoc-Pro-OH
Fmoc-Ser(tBu)-OH
Fmoc-Thr-(tBu)
Fmoc-Tyr(tBu)-OH
Fmoc-Val-OH
Fmoc-Lys-(Fmoc)—OH

### Example 2: Immunizations

Syrian golden hamsters were immunized on day 1 and 28, with approximately 70 μg of each peptide in 250 μL of adjuvant with adjuvanted vaccines containing the five polypeptides. Groups of five animals were immunized subcutaneously on days 0 and 28. Serum was collected and data from IgG blots for days 0, 27, 48, and 120 bleeds are shown below. A nitrocellulose blot technique for proteins was developed and used in this study. The proteins were the spike proteins of 2019 Wuhan, Delta, and Omicron BA. 1 variants of SARS-CoV-2. These proteins were utilized to detect IgG antibodies in groups of five animals with the Vitamin E adjuvanted nonphospholipid-based liposome.

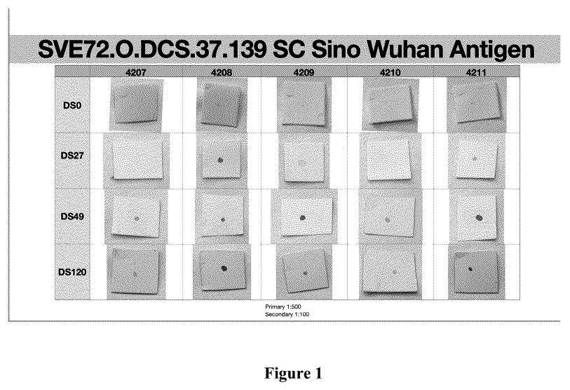

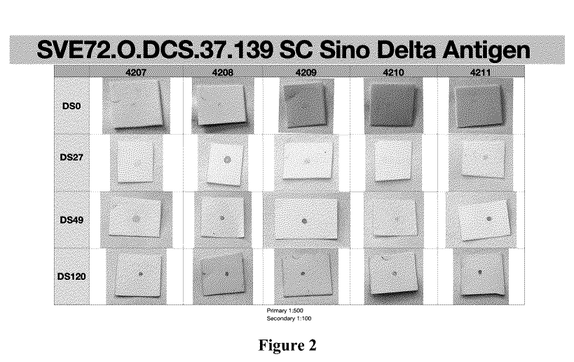

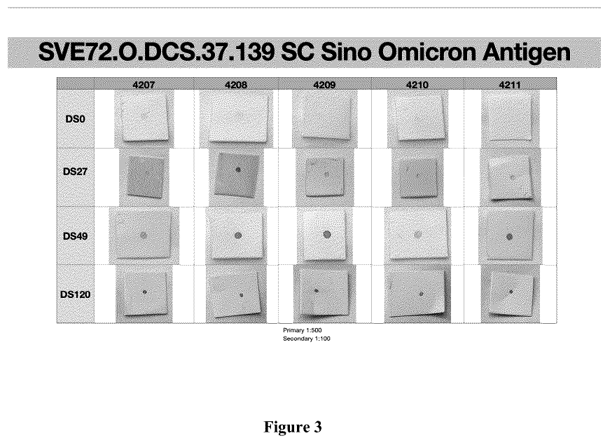

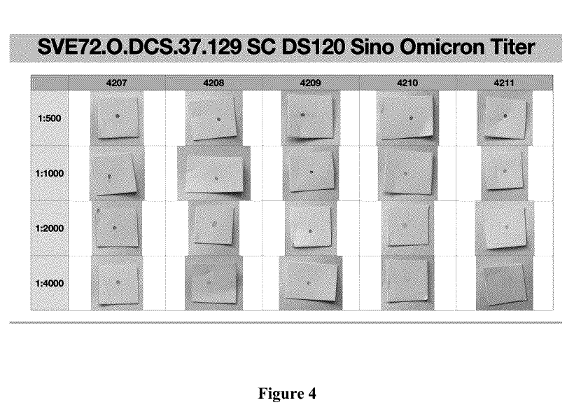

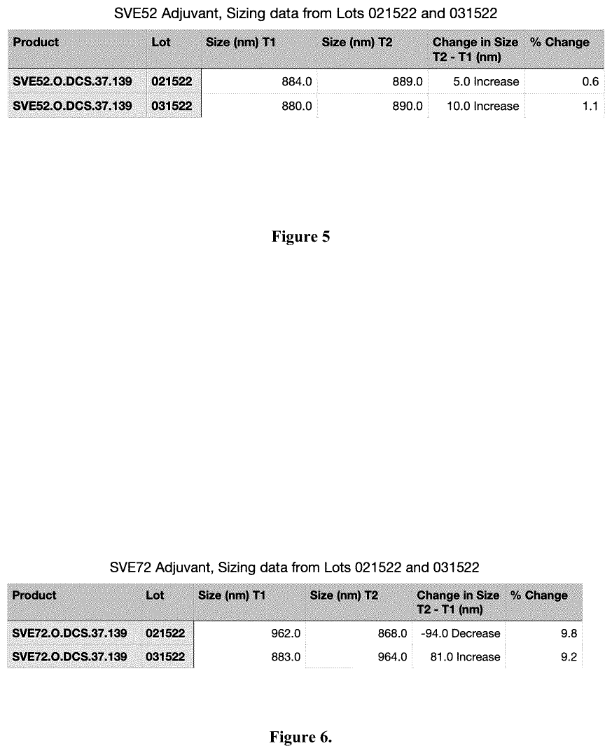

SVE are the initials for the Vitamin E-containing nonphospholipid-based liposome adjuvant. All animals were immunized subcutaneously. FIGS. 1, 2, and 3 show the bleed test data. Blot images show IgG antibodies directed against the spike proteins of 2019 Wuhan, Delta, and Omicron BA. 1 variants of SARS-CoV-2.

The adjuvant formulations used for the above experiments were prepared using a reciprocating syringe technique which produced 5 milliliters of adjuvanted polypeptide.

The lipid formulations were composed of polyoxyethylene-2-stearyl-ether (28.10 g), cholesterol (10.8 g), Vitamin E (5.4 g), and oleic acid (120 μl), or polyoxyethylene-2-cetyl-ether (30.01 g), cholesterol (12.80 g), Vitamin E (6.00 g), and oleic acid (125 μl). Polypeptides were solubilized in sterile water for injection at a concentration of 1.25 mg/mL. The lipid:diluent ratio on mixing was 1:4 on a volume basis. The final concentration of polypeptide in the adjuvanted vaccine was 1.0 mg peptide in 1.0 g adjuvant.

SEQUENCE ID NO: 1
surface glycoprotein [Severe acute respiratory syndrome coronavirus 2]
GenBank: UGO97992.1
Identical Proteins FASTA Graphics
Go to:

| | |
|---|---|
| LOCUS | UGO97992 1267 aa linear VRL 11-DEC-2021 |
| DEFINITION | surface glycoprotein [Severe acute respiratory syndrome |
| | coronavirus 2]. |
| ACCESSION | UGO97992 |
| VERSION | UGO97992.1 |
| DBSOURCE | accession OL815451.1 |
| KEYWORDS | . |
| SOURCE | Severe acute respiratory syndrome coronavirus 2 (SARS-CoV-2) |
| ORGANISM | Severe acute respiratory syndrome coronavirus 2 |
| | Viruses; Riboviria; Orthornavirae; Pisuviricota; |
| | Pisoniviricetes; Nidovirales; Cornidovirineae; Coronaviridae; |
| | Orthocoronavirinae; Betacoronavirus; Sarbecovirus. |
| REFERENCE | 1 (residues 1 to 1267) |
| AUTHORS | Howard, D., Batra, D., Cook, P. W., Caravas, J., Rambo-Martin, B., |
| | Sammons, S., Unoarumhi, Y., Schmerer, M., Lacek, K.A., Kendall, T., |
| | Caban Figueroa, V., Morrison, S., Gulvick, C., Sula, E., Gao, H., |
| | Li, M., |
| | Gao, J., Fierro, J., Sapra, B., Tsai, B., Meng, Y., Ng, D., Xie, J., |
| | Paden, C.R. and MacCannell, D. |
| TITLE | CDC Sars CoV2 Sequencing Baseline Constellation |
| JOURNAL | Unpublished |
| REFERENCE | 2 (residues 1 to 1267) |
| AUTHORS | Howard, D., Batra, D., Cook, P. W., Caravas, J. , Rambo-Martin, B., |
| | Sammons, S., Unoarumhi, Y., Schmerer, M., Lacek, K.A., Kendall, T., |
| | Caban Figueroa, V., Morrison, S., Gulvick, C., Sula, E., Gao, H., |
| | Li, M., |
| | Gao, J., Fierro, J., Sapra, B., Tsai, B., Meng, Y., Ng, D., Xie, J., |
| | Paden, C.R. and MacCannell, D. |
| TITLE | Direct Submission |
| JOURNAL | Submitted (10-DEC-2021) Respiratory Viruses Branch, Division of |
| | Viral Diseases, Centers for Disease Control and Prevention, |
| | 1600 |
| | Clifton Rd, Atlanta, GA 30329, USA |
| COMMENT | Method: conceptual translation. |
| FEATURES | Location/Qualifiers |
| source | 1..1267 |
| | /organism = ″Severe acute respiratory syndrome |
| | coronavirus |
| | 2″ |
| | /isolate = ″SARS-COV-2/human/USA/CA-CDC-FG-180156/2021″ |
| | /isolation source = ″nasal swab″ |
| | /host = ″Homo sapiens″ |
| | /db_xref = ″taxon: 2697049″ |
| | /country = ″USA: California″ |
| | /collection date = ″2021-11-30″ |
| Protein | 1..1267 |
| | /product = ″surface glycoprotein″ |
| CDS | 1..1267 |
| | /gene = ″S″ |
| | /coded_by = ″OL815451.1:21514..25317″ |

ORIGIN

| | |
|---|---|
| 1 | mfvflvllpl vssqcvnltt rtqlppaytn sftrgvyypd kvfrssvlhs tqdlflpffs |
| 61 | nvtwfhvisg tngtkrfdnp vlpfndgvyf asieksniir gwifgttlds ktqsllivnn |
| 121 | atnvvikvce fqfcndpfld hknnkswmes efrvyssann ctfeyvsqpf lmdlegkqgn |
| 181 | fknlrefvfk nidgyfkiys khtpiivrdl pggfsalepl vdlpiginit rfqtllalhr |
| 241 | syltpgdsss gwtagaaayy vgylqprtfl lkynengtit davdcaldpl setkctlksf |
| 301 | tvekgiyqts nfrvqptesi vrfpnitnlc pfdevfnatr fasvyawnrk risncvadys |
| 361 | vlynlapfft fkcygvsptk lndlcftnvy adsfvirgde vrqiapgqtg niadynyklp |
| 421 | ddftgcviaw nsnkldskvs gnynylyrlf rksnlkpfer disteiyqag nkpcngvagf |
| 481 | ncyfplrsys frptygvghq pyrvvvlsfe llhapatvcg pkkstnlvkn kcvnfnfngl |
| 541 | kgtgvltesn kkflpfqqfg rdiadttdav rdpqtleild itpcsfggvs vitpgtntsn |
| 601 | qvavlyqgvn ctevpvaiha doltptwrvy stgsnvfqtr agcligaeyv nnsyecdipi |
| 661 | gagicasyqt qtkshrrars vasqsiiayt mslgaensva ysnnsiaipt nftisvttei |
| 721 | lpvsmtktsv dctmyicgds tecsnlllqy gsfctqlkra ltgiavegdk ntqevfaqvk |
| 781 | qiyktppiky fggfnfsqil pdpskpskrs fiedllfnkv tladagfikq ygdclgdiaa |
| 841 | rdlicaqkfk gltvlppllt demiagytsa llagtitsgw tfgagaalqi pfamqmayrf |
| 901 | ngigvtqnvl yenqklianq fnsaigkiqd slsstasalg klqdvvnhna qalntlvkql |
| 961 | sskfgaissv lndifsrldk veaevqidrl itgrlqslqt yvtqqliraa eirasanlaa |
| 1021 | tkmsecvlgq skrvdfcgkg yhlmsfpqsa phgvvflhvt yvpageknft tapaichdgk |
| 1081 | ahfpregvfv sngthwfvtq rnfyepqiit tdntfvsgnc dvvigivnnt vydplqpeld |
| 1141 | sfkeeldkyf knhtspdvdl gdisginasv vniqkeidrl nevaknlnes lidlqelgky |
| 1201 | egyikwpwyi wlgfiaglia ivmvtimlcc mtsccsclkg ccscgscckf deddsepvlk |
| 1261 | gvklhyt |

SEQUENCE ID NO: 2
SARS-CoV-2 mutations from new variant B.1.1.529 Omicron 71 isolates in South Africa
Nov. 11, 2021 superimposed on the surface glycoprotein [SARS-CoV-2, China,
Human Isolate, 2/18/20]: QJG65958.1
mfvflvllplvssqcvnlttrtqlppaytnsftrgvyypdkvfrssvlhstqdlflpffs
nvtwfhaihvsgtngtkrfdnpvlpfndgvyfasteksniirgwifgttldsktqslliv
nnatnvvikvcefqfcndpflgvyyhknnkswmesefrvyssannctfeyvsqpflmdle
gkqgnfknlrefvfknidgyfkiyskhtpinlveperdlpggfsaleplvdlpiginitrfqt
llalhrsyltpgdsssgwtagaaayyvgylqpmtyilkynengtitdavdcaldplsetk
ctlksftvekgiyqtsnfrvqptesivrfpnitnlcpfgevfnatrfasvyawnrkrisn
cvadysvlynsasfstfkcygvsptklndlcftnvyadsfvirgdevrqiapgqtgkiad
ynyklpddftgcviawnsnnldskvggnynylyrlfrksnlkpferdisteiyqagstpc
ngvegfncyfplqsygfqptngvgyqpyrvvvlsfellhapatvcgpkkstnlvknkcvn
fnfngltgtgvltesnkkflpfqqfgrdiadttdavrdpqtleilditpcsfggvsvitp
gtntsnqvavlygdvnctevpvaihadgltptwrvystgsnvfqtragcligaehvnnsy
ecdipigagicasyqtqtnsprrarsvasqsiiaytmslgaensvaysnnsiaiptnfti
svtteilpvsmtktsvdctmyicgdstecinlllqygsfctqlnraltgiaveqdkntqe
vfaqvkqiyktppikdfggfnfsqilpdpskpskrsfiedllfnkvtladagfikqygdc
lgdiaardlicaqkfngltvlpplltdemiaqytsallagtitsgwtfgagaalqipfam
qmayrfngigvtqnvlyengklianqfnsaigkiqdslsstasalgklqdvvnqnaqaln
tlvkqlssnfgaissvlndilsrldkveaevqidrlitgrlqslqtyvtqqliraaeira
sanlaatkmsecvlgqskrvdfcgkgyhlmsfpqsaphgvvflhvtyvpageknfttapa
ichdgkahfpregvfvsngthwfvtqrnfyepgiittdntfvsgncdvvigivnntvydp
lgpeldsfkeeldkyfknhtspdvdlgdisginasvvniqkeidrlnevaknlneslidl
qelgkyegyikwpwyiwlgfiagliaivmvtimlccmtsccsclkgccscgscckfdedd
sepvlkgvklhyt
SEQUENCE ID NO: 3
mfvflvllplvssqcvnlttrtqlppaytnsftrgvyypdkvfrssvlhstqdlflpffs
nvtwfhaihvsgtngtkrfdnpvlpfndgvyfasteksniirgwifgttldsktqslliv
nnatnvvikvcefqfcndpflgvyyhknnkswmesefrvyssannctfeyvsqpflmdle
gkqgnfknlrefvfknidgyfkiyskhtpinlveperdlpqgfsaleplvdlpiginitrfqt
llalhrsyltpgdsssgwtagaaayyvgylqpmtyilkynengtitdavdcaldplsetk
ctlksftvekgiyqtsnfrvqptesivrfpnitnlcpfgevfnatrfasvyawnrkrisn
cvadysvlynsasfstfkcygvsptklndlcftnvyadsfvirgdevrgiapgqtgkiad
ynyklpddftgcviawnsnnldskvggnynylyrlfrksnlkpferdisteiygagstpc
ngvegfncyfplqsygfqptngvgyqpyrvvvlsfellhapatvcgpkkstnlvknkcvn
fnfngltgtgvltesnkkflpfqqfgrdiadttdavrdpqtleilditpcsfggvsvitp
gtntsnqvavlyqdvnctevpvaihadqltptwrvystgsnvfqtragcligaehvnnsy
ecdipigagicasyqtqtnsprrarsvasqsiiaytmslgaensvaysnnsiaiptnfti
svtteilpvsmtktsvdctmyicgdstecinlllqygsfctqlnraltgiaveqdkntge
vfaqvkqiyktppikdfggfnfsqilpdpskpskrsfiedllfnkvtladagfikqygdc
lgdiaardlicaqkfngltvlpplltdemiaqytsallagtitsgwtfgagaalqipfam
qmayrfngigvtqnvlyenqklianqfnsaigkiqdslsstasalgklqdvvngnagaln
tlvkqlssnfgaissvlndilsrldkveaevqidrlitgrlqslqtyvtqqliraaeira
sanlaatkmsecvlgqskrvdfcgkgyhlmsfpqsaphgvvflhvtyvpaqeknfttapa
ichdgkahfpregvfvsngthwfvtgrnfy
SEQUENCE ID NOS: 4-15: Amino Acid Sequences of Twelve Polypeptides from Modified
Omicron BA.1 Sequence (1106 amino acids)
SEQ ID NO: 4
mfvflvllpl vssqcvnltt rtqlppaytn sftrgvyypd kvfrssvlhs
tqdlflpffs nvtwfhvi sgtngtkrfd npvlpfndgv yfasieksni
SEQ ID NO: 5
irgwifgttl dsktqslliv nnatnvvikv cefqfcndpf ldhknnk swmesefrvy
ssannctfey vsqpflmdle gkqgnfknlr efvfknidgy
SEQ ID NO: 6
fkiyskhtpi iveperdlpqgf saleplvdlp iginitrfqt llalhrsylt
pgdsssgwta gaaayyvgyl qpmtyilkyn engtitdavd caldplsetk ctlk
SEQ ID NO: 7
sftvek giyqtsnfrv qptesivrfp nitnlcpfde vfnatrfasv yawnrkrisn
cvadysvlyn lapfftfkcy gvsptkindl cftnvyadsf
SEQ ID NO: 8
virgdevrqi apgqtgniad ynyklpddft gcviawnsnk lskvsgnyn
ylyrlfrksn lkpferdist eiyqagnkpc ngvagincyf plksysfrpt
ygvghqpyrv
SEQ ID NO: 9
vvlsfellha patvcgpkks tnlvknkcvn nfnglkgtg vltesnkkfl
pfqqfgrdia dttdavrdpq tleilditpc sfggvsvitp
SEQ ID NO: 10
gtntsnqvav lyqgvnctev pvaihadqlt ptwrvystgs nvfqtragcl
aensvaysnn ecdipigagi casyqtqtks hrrarsvasq siiaytmslg
igaeyvnnsy
SEQ ID NO: 11
siaipnnfti svtteilpvs mtktsvdctm yicgdsteci nlllqygsfc
tqlkraltgi aveqdkntqe vfaqvkqiyk tppikyfggf nfsqilpdps
SEQ ID NO: 12
kpskrsfied llfnkvtlad agfikqygdc lgdiaardli caqkfkgltv
lpplltdemi aqytsallag titsgwtfga gaalqipfam qmayringig v
SEQ ID NO: 13
tqnvlyenq klianqfnsa igkiqdslss tasalgklqd vvnhnaqaln
tlvkqlsskf gaissvlndi fsrldkveae
SEQ ID NO: 14
vqidrlitgr lqslqtyvtq qliraaeira sanlaatkms ecvlgqskrv
dfcgkgyhlm sfpqsaphgv vflhvtyvpa
SEQ ID NO: 15
qeknfttapa ichdgkahfp regvfvsngt hwfvtqrnfy
SEQUENCE ID NOS: 16-20: Five polypeptides derived from SARS-CoV-2
Omicron Spike Protein of B.1.1.529, containing a total of 323 amino
acids
SEQ ID NO: 16CS18006 D-801 DCS.O.37
Asn-Asn-Ala-Thr-Asn-Val-Val-Ile-Lys-Val-Cys-Glu-Phe-Gln-Phe-Cys-Asn-Asp-Pro-
Phe-Leu-Asp-His-
Lys-Asn-Asn-Lys-Ser-Trp-Met-Glu-Ser-Glu-Phe-Arg-Val-Tyr-NH₂ [Linear Peptide]
nnatnvvikv cefqfondpf ldhknnk swmese frvy
SEQ ID NO: 17
CS18007 D-802 DCS.O.60
Glu-Ile-Tyr-Gln-Ala-Gly-Asn-Lys-Pro-Cys-Asn-Gly-Val-Ala-Gly-Phe-Asn-Cys-Tyr-
Phe-Pro-Leu-Lys-Ser-
Tyr-Ser-Phe-Arg-Pro-Thr-Tyr-Gly-Val-Gly-His-Gln-Pro-Tyr-Arg-Val-Val-Val-Leu-Ser-
Phe-Glu-Leu-Leu-
His-Ala-Pro-Ala-Thr-Val-Cys-Gly-Pro-Lys-Lys-Ser-NH₂ [Linear Peptide]
elyqagnkpc ngvagfncyf plksysfrpt ygvghqpyrv vvlsfellha patvcgpkks
SEQ ID NO: 18
CS18008 D-803 DCS.O.96
Ser-Phe-Thr-Val-Glu-Lys-Gly-Ile-Tyr-Gln-Thr-Ser-Asn-Phe-Arg-Val-Gln-Pro-Thr-Glu-
Ser-Ile-Val-Arg-
Phe-Pro-Asn-Ile-Thr-Asn-Leu-Cys-Pro-Phe-Asp-Glu-Val-Phe-Asn-Ala-Thr-Arg-Phe-
Ala-Ser-Val-Tyr-Ala-
Trp-Asn-Arg-Lys-Arg-Ile-Ser-Asn-Cys-Val-Ala-Asp-Tyr-Ser-Val-Leu-Tyr-Asn-Leu-
Ala-Pro-Phe-Phe-Thr-
Phe-Lys-Cys-Tyr-Gly-Val-Ser-Pro-Thr-Lys-Leu-Asn-Asp-Leu-Cys-Phe-Thr-Asn-Val-
Tyr-Ala-Asp-Ser-
Phe-NH₂ [Linear Peptide]
sftvek giyqtsnfrv qptesivrfp nitnlcpfde vfnatrfasv yawnrkrisn cvadysvlyn
lapfftfkcy gvsptklndl cftnvyadsf
SEQ ID NO: 19
CS18009 D-804 DCS.O.40
Val-Val-Asn-His-Asn-Ala-Gln-Ala-Leu-Asn-Thr-Leu-Val-Lys-Gln-Leu-Ser-Ser-Lys-
Phe-Gly-Ala-Ile-Ser-
Ser-Val-Leu-Asn-Asp-Ile-Phe-Ser-Arg-Leu-Asp-Lys-Val-Glu-Ala-Glu-NH₂
vvnhnagain tlvkqlsskf gaissvindi fsrldkveae
SEQ ID NO: 20
CS18010 D-805 DCS.O.90
Gly-Thr-Asn-Thr-Ser-Asn-Gln-Val-Ala-Val-Leu-Tyr-Gln-Gly-Val-Asn-Cys-Thr-Glu-
Val-Pro-Val-Ala-Ile-
His-Ala-Asp-Gln-Leu-Thr-Pro-Thr-Trp-Arg-Val-Tyr-Ser-Thr-Gly-Ser-Asn-Val-Phe-
Gln-Thr-Arg-Ala-Gly-
Cys-Leu-Ile-Gly-Ala-Glu-Tyr-Val-Asn-Asn-Ser-Tyr-Glu-Cys-Asp-Ile-Pro-Ile-Gly-Ala-
Gly-Ile-Cys-Ala-
Ser-Tyr-Gln-Thr-Gln-Thr-Lys-Ser-His-Arg-Arg-Ala-Arg-Ser-Val-Ala-Ser-Gln-NH₂
[Linear Peptide]
gtntsnqvav lyqgvnctev pvaihadqlt ptwrvystgs nvfqtragcl igaeyvnnsyecdipigagi
casyqtqtks hrrarsvasq

1. A vaccine comprising at least five polypeptides and a non-phospholipid liposome, wherein the liposome contains Vitamin E.

2. The vaccine of claim 1 wherein the liposome comprises between 2-10 bilayers surrounding an amorphous central cavity, and wherein said nonphospholipid materials are selected from the group consisting of polyoxyethylene fatty acid esters, polyoxyethylene fatty acid ethers, polyoxyethylene sorbitan esters, polyoxyethylene glyceryl mono- and diesters, glyceryl mono- and distearate, sucrose distearate, propylene glycol stearate, long chain acyl hexosamides, long chain acyl amino acid amides, long chain acyl amides, glyceryl mono- and diesters, dimethyl acyl amines, C 12-C 20 fatty alcohols, C 12-C 20 glycol monoesters, and C 12-C 20 fatty acids.

3. The vaccine of claim 1 wherein the at least five polypeptides are encapsulated within the liposome.

4. The vaccine of claim 1 wherein said antigen is encapsulated within the amorphous central cavity of the liposome.

5. The vaccine of claim 1 wherein five polypeptides are selected from the group of twelve polypeptides representing the majority of the Spike protein of SARS-CoV-2 variant Omicron BA.1, consisting of at least five of SEQ ID NOS: 4-15.

6. The vaccine of claim 1 wherein the liposome further comprises at least one sterol selected from the group consisting of cholesterol or cholesterol derivatives.

7. The vaccine of claim 1 wherein the liposome comprises an amorphous central cavity containing Vitamin E.

8. The vaccine of claim 1, wherein the vaccine generates antibodies that recognize Omicron spike protein and the polypeptide construct.

9. A vaccine comprising five polypeptide antigens and a non-phospholipid liposome, wherein the vaccine generates antibodies that recognize the Spike protein of the 2019 Wuhan SARS-CoV-2 isolate, the SARS-CoV-2 Delta variant, and the SARS-CoV-2 Omicron BA.1 variant.

10. The vaccine of claim 1, wherein at least five polypeptides are derived from SARS-CoV-2 isolates already identified, e.g. the Wuhan isolates, Alpha, Beta, Gamma, Delta, Omicron, and any future variants of SARS-CoV-2.

11. The vaccine of claim 1, wherein at least five polypeptides are selected from SEQ ID NOS: 4-15.

12. A subcutaneously-injected, penta-polypeptide, nonphospholipid-based liposome vaccine that after two injections separated by 28 days generates a significant IgG response by blot against the 2019 Wuhan spike protein, the Delta variant, and the Omicron BA. 1 variant.

13. A subcutaneously-injected, penta-polypeptide, nonphospholipid-based liposome vaccine that after two injections separated by 28 days generates a significant IgG response by blot at day 120, 71 days after the second dose, against the Spike protein of the 2019 Wuhan isolate, the Delta variant, and the Omicron BA. 1 variant.

14. (canceled)

15. (canceled)
